(19) United States
(12) Patent Application Publication
(10) Pub. No.: US 2006/0110416 A1
(43) Pub. Date: May 25, 2006

(54) Hydroalcoholic cosmetic compositions

(75) Inventors: Christine Watson Ryles, Milford, CT (US); Paul Vinski, Danbury, CT (US); Alexander George Ziemkiewicz, Shelton, CT (US); Kenneth Paul Manzari, West Haven, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/995,760

(22) Filed: Nov. 23, 2004

(51) Int. Cl. A61K 8/34

(52) U.S. Cl. 424/401

(57) ABSTRACT

A hydroalcoholic cosmetic composition is provided which when topically applied to the face can shrink skin pores. The composition is focused upon denatured ethyl alcohol utilizing menthol as denaturing agent. Further included in the composition are humectants, preservatives and water. Also described is a method for reducing skin pore size.

### BACKGROUND OF THE INVENTION

1. Field of the Invention

The invention relates to hydroalcoholic cosmetic compositions and their use in shrinking facial pores.

2. The Related Art

Women have many concerns with their facial complexion. Causes for concern are fine lines and wrinkles, hyperpigmentation, elasticity and pore problems. Some remedies such as alpha hydroxy acids and retinol can address aging and sun damage imperfections. Application of these actives can smooth fine lines and wrinkles and diminish hyperpigmentation spots. Shrinkage of pores has received less attention. As a result, the only well-known remedy is covering up the imperfection under a coating of facial foundation or other opaque skin product.

The literature has disclosed hydroalcoholic solutions formulated to remove pimples and red spots. U.S. Pat. No. 5,482,710 (Slavtcheff et al.) formulates hydroalcoholic solutions that combine an alpha hydroxy carboxylic acid, bisabolol and glycyrrhizinate salts.

Despite many earlier attempts, there have been no significant solutions to the problem of shrinking skin pore sizes.

### SUMMARY OF THE INVENTION

A hydroalcoholic cosmetic composition is provided which includes:
(i) from about 1 to about 60% denatured ethyl alcohol by weight of the composition and containing from about 0.3 to about 3% menthol by weight of the alcohol;
(ii) from about 0.5 to about 80% humectant by weight of the composition;
(iii) from about 0.00001 to about 3% preservative by weight of the composition; and
(iv) from about 10% to about 85% water by weight of the composition.

### DETAILED DESCRIPTION OF THE INVENTION

Now it has been found that hydroalcoholic cosmetic compositions utilizing ethyl alcohol denatured with menthol promotes shrinkage of skin pores when applied to the skin.

Ordinarily the ethyl alcohol will be SDA 38B grade which has been denatured with menthol or a menthol containing mixture such as peppermint oil.

Amounts of the denatured ethyl alcohol may range from about 1 to about 60%, preferably from about 10 to about 40%, optimally from about 12 to about 20% by weight of the composition. Denaturizing amounts of menthol in the ethyl alcohol will range from about 0.3 to about 3%, preferably from about 1 to about 2.5%, and optimally from about 1.3 to about 2% by weight of the denatured ethyl alcohol.

Water will be present in compositions of this invention. Amounts of water may range from about 5 to about 95%, preferably from about 20 to about 85%, more preferably from about 30 to about 75%, and optimally from about 40% to about 70% by weight of the composition.

Humectants are another element of the present invention. This category is represented by polyhydric alcohol-type materials. Illustrative are glycerol (also known as glycerin), propylene glycol, dipropylene glycol, polypropylene glycol, polyethylene glycol, sorbitol, hydroxypropyl sorbitol, hexylene glycol, 1,3-butylene glycol, 1,2,6-hexane triol, ethoxylated glycerol, propoxylated glycerol, maltitol, isoprene glycol and mixtures thereof.

Preservatives are components of the present invention. Particularly preferred preservatives are phenoxyethanol, methyl paraben, propyl paraben, imidazolidinyl urea, dimethyloldimethylhydantoin, ethylenediaminetetraacetic acid salts (EDTA), sodium dehydroacetate, methylchloroisothiazolinone, methylisothiazolinone, iodopropynbutylcarbamate and benzyl alcohol. The preservatives should be selected having regard for the use of the composition and possible incompatibilities between the preservatives and other ingredients. Preservatives are preferably employed in amounts ranging from about 0.00001 to about 3%, preferably from about 0.01% to about 2% by weight of the composition.

Cosmetic compositions of this invention may be cleansers, toners, creams, gels, serums, masks and lotions. The term “cosmetic” means any composition which provides a benefit to the skin, and excludes compositions such as toothpaste and mouthwash used in the oral cavity. Most particularly, the hydroalcoholic compositions of this invention are in toner format. In these compositions it is desirable to have a solubilizer. Illustrative solubilizers are polypropylene glycol copolymers with C₈-C₂₄ fatty acids or alcohols. A particular example is PPG-2 Isoceteth-20 Acetate. Amounts of the solubilizer may range from about 0.1 to about 20%, preferably from about 0.5 to about 1% by weight of the composition.

When in toner form, the composition advantageously is a transparent or translucent gel which maintains suspended bubbles through use of the solubilizer. The bubbles have an average diameter ranging from about 0.001 to about 2 mm and range in amount from about 1 to about 1,000 per cc³. Viscosity of the gel ranges from about 2,000 to about 15,000, preferably from about 5 to about 8,000 cp as measured on a Brookfield RVF Viscometer at 10 rpm using Spindle 5 at 25° C. The bubbles of gas (e.g. air, nitrogen or carbon dioxide) in the gel are stable at 25° C. for at least 7 days.

Compositions of this invention may include a variety of other functional ingredients. These include emollients, fatty acids, fatty alcohols, thickeners and combinations thereof.

Emollient materials may be in the form of silicone oils, natural or synthetic esters and hydrocarbons. Amounts of the emollients may range anywhere from about 0.1 to about 60%, preferably from about 1 and about 30% by weight of the composition.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁶ to 0.1 m²/s at 25° C. Among the preferred nonvolatile emollients useful in the present compositions are the polydimethyl siloxanes having viscosities from about 1×10⁻⁵ to about 4×10⁻⁴ m²/s at 25° C.

Another class of nonvolatile silicones are emulsifying and non-emulsifying silicone elastomers. Representative of this category is DimethiconeNinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:

a) Alkyl esters of saturated fatty acids having 10 to 24 carbon atoms. Examples thereof include behenyl neopentanoate, isononyl isonanonoate, isopropyl myristate and octyl stearate.

b) Ether-esters such as fatty acid esters of ethoxylated saturated fatty alcohols.

c) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

d) Wax esters such as beeswax, spermaceti wax and tribehenin wax.

e) Sugar ester of fatty acids such as sucrose polybehenate and sucrose polycotton seed ate.

Natural ester emollients principally are based upon mono-, di- and tri-glycerides. Representative glycerides include sunflower seed oil, cottonseed oil, borage oil, borage seed oil, primrose oil, castor and hydrogenated castor oils, rice bran oil, soybean oil, olive oil, safflower oil, shea butter, jojoba oil and combinations thereof. Animal derived emollients are represented by lanolin oil and lanolin derivatives. Amounts of the natural esters may range from about 0.1 to about 20% by weight of the compositions.

Hydrocarbons which are suitable include petrolatum, mineral oil, C₁₁-C₁₃ isoparaffins, polybutenes, and especially isohexadecane, available commercially as Permethyl 101A from Presperse Inc.

Fatty acids having from 10 to 30 carbon atoms may also be present. Illustrative of this category are pelargonic, lauric, myristic, palmitic, stearic, isostearic, oleic, linoleic, linolenic, hydroxystearic and behenic acids.

Fatty alcohols having from 10 to 30 carbon atoms are another useful category of ingredient. Illustrative are stearyl alcohol, lauryl alcohol, myristyl alcohol, oleyl alcohol and cetyl alcohol.

Thickeners can be utilized as part of compositions according to the present invention. Typical thickeners include crosslinked acrylates (e.g. Carbopol 982®), hydrophobically-modified acrylates (e.g. Carbopol 1382®), polyacrylamides (e.g. Sepigel 305®), acryloylmethylpropane sulfonic acid/salt polymers and copolymers (e.g. Aristoflex HMB® and AVC®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, talc, calcium carbonate and silicates such as magnesium aluminum silicate (Veegum®). Amounts of the thickener may range from 0.0001 to 10%, usually from 0.001 to 2%, optimally from 0.01 to 1.5% by weight of the composition.

Surfactants may also be present in compositions of the present invention. Total concentration of the surfactant when present may range from about 0.1 to about 90%, preferably from about 1 to about 40%, optimally from about 1 to about 20% by weight of the composition, and being highly dependent upon the type of cosmetic product. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) and trialkylamine oxides are also suitable nonionic surfactants.

Preferred anionic surfactants include soap, alkyl ether sulfates and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, C₈-C₂₀ sarcosinates, C₈-C₂₀ acyl lactylates, sulfoacetates and combinations thereof.

Useful amphoteric surfactants include cocoamidopropyl betaine, cocoamidopropyl hydroxysultaine, C₁₂-C₂₀ trialkyl betaines, sodium lauroamphoacetate, and sodium laurodiamphoacetate.

Sunscreen agents may also be included in compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide and zinc oxide. Amounts of the sunscreen agents when present may generally range from 0.1 to 30%, preferably from 2 to 20%, optimally from 4 to 10% by weight of the composition.

Antiperspirants and deodorant compositions of the present invention ordinarily will contain astringent actives. Examples include aluminum chloride, aluminum chlorhydrex, aluminum-zirconium chlorhydrex glycine, aluminum sulfate, zinc sulfate, zirconium and aluminum chlorohydroglycinate, zirconium hydroxychloride, zirconium and aluminum lactate, zinc phenolsulfonate and combinations thereof. Amounts of the astringents may range anywhere from about 0.5 to about 50% by weight of the composition.

Compositions of the present invention may include vitamins. Illustrative vitamins are Vitamin A (retinol), Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present in compositions according to the present invention may range from 0.001 to 10%, preferably from 0.01% to 1%, optimally from 0.1 to 0.5% by weight of the composition.

Another type of useful substance can be that of an enzyme such as amylases, oxidases, proteases, lipases and combinations. Particularly preferred is superoxide dismutase, commercially available as Biocell SOD from the Brooks Company, USA.

Skin lightening compounds may be included in the compositions of the invention. Illustrative substances are placental extract, lactic acid, niacinamide, arbutin, kojic acid, ferulic acid, resorcinol and derivatives including 4-substituted resorcinols and combinations thereof. Amounts of these agents may range from about 0.1 to about 10%, preferably from about 0.5 to about 2% by weight of the composition.

Desquamation promoters may be present. Illustrative are the alpha-hydroxycarboxylic acids and beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic, lactic and malic acids. Salicylic acid is representative of the beta-hydroxycarboxylic acids. Amounts of these materials when present may range from about 0.01 to about 15% by weight of the composition.

A variety of herbal extracts may optionally be included in compositions of this invention. The extracts may either be water soluble or water-insoluble carried in a solvent which respectively is hydrophilic or hydrophobic. Water and ethanol are the preferred extract solvents. Illustrative extracts include those from green tea, chamomile, licorice, aloe vera, grape seed, citrus unshui, willow bark, sage, thyme and rosemary.

Also included may be such materials as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials may range from about 0.000001 to about 10%, preferably from about 0.0001 to about 1% by weight of the composition.

Colorants, opacifiers and abrasives may also be included in compositions of the present invention. Each of these substances may range from about 0.05 to about 5%, preferably between 0.1 and 3% by weight of the composition.

The compositions of the present invention can also be, optionally, incorporated into an insoluble substrate for application to the skin such as in the form of a treated wipe.

A wide variety of packaging can be employed to store and deliver the cosmetic compositions. Packaging is often dependent upon the type of personal care end-use. For instance, leave-on skin lotions and creams generally employ plastic containers with an opening at a dispensing end covered by a closure. Typical closures are screw-caps, non-aerosol pumps and flip-top hinged lids. Packaging for antiperspirants and deodorants involve a container with a roll-on ball on a dispensing end. Metallic cans pressurized by a propellant and having a spray nozzle may serve as packaging for antiperspirants and other personal care products. All of the aforementioned are considered packaging within context of the present invention.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

All documents referred to herein, including all patents, patent applications, and printed publications, are hereby incorporated by reference in their entirety in this disclosure.

The following examples will more fully illustrate the embodiments of this invention. All parts, percentages and proportions referred to herein and in the appended claims are by weight unless otherwise illustrated.

### EXAMPLE 1

This Example illustrates a gel-type toner according to the present invention. Table I outlines illustrative toners.

TABLE I

| | Sample (Weight %) | | | | |
|---|---|---|---|---|---|
| Ingredient | 1A | 1B | 1C | 1D | 1E |
| SD Alcohol 38B (Menthol | 10 | 15 | 20 | 25 | 30 |
| Denatured) | | | | | |
| Dipropylene Glycol | 5 | 5 | 5 | 5 | 5 |
| Glycerin | 1.5 | 1.5 | 1.5 | 1.5 | 1.5 |
| Witch hazel | 1 | 1 | 1 | 1 | 1 |
| PPG-2 Isoceteth-20 Acetate | 0.6 | 0.6 | 0.6 | 0.6 | 0.6 |
| Triethanolamine | 0.3 | 0.3 | 0.3 | 0.3 | 0.3 |
| Carbopol Ultrez 10 ® | 0.2 | 0.2 | 0.2 | 0.2 | 0.2 |
| Methyl Paraben | 0.2 | 0.2 | 0.2 | 0.2 | 0.2 |
| DMDM Hydantoin | 0.3 | 0.3 | 0.3 | 0.3 | 0.3 |
| Fragrance | 0.1 | 0.1 | 0.1 | 0.1 | 0.1 |
| DL Panthenol | 0.1 | 0.1 | 0.1 | 0.1 | 0.1 |
| Vitamin A Palmitate | 0.01 | 0.01 | 0.01 | 0.01 | 0.01 |
| Vitamin E Acetate | 0.01 | 0.01 | 0.01 | 0.01 | 0.01 |
| Water | Bal. | Bal. | Bal. | Bal. | Bal. |

### EXAMPLE 2

This Example illustrates moisturizer creams. Table II reports components of various related moisturizer compositions.

TABLE II

| | Sample (Weight %) | | | | |
|---|---|---|---|---|---|
| Ingredient | 2A | 2B | 2C | 2D | 2E |
| Cyclopentasiloxane | 10.75 | 9.50 | 9.50 | 8.25 | 12.50 |
| SD Alcohol 38B (Menthol | 3.00 | 3.00 | 6.00 | 6.00 | 2.00 |
| Denatured) | | | | | |
| Glycerin | 2.50 | 3.00 | 2.50 | 3.50 | 1.75 |
| Butylene Glycol | 2.00 | 2.00 | 2.00 | 2.00 | 2.00 |
| Shea Butter | 2.00 | 2.00 | 2.00 | 2.00 | 2.00 |
| Caprylic/Capric Triglycerides | 5.70 | 5.70 | 5.70 | 5.70 | 5.70 |
| Aristoflex AVC ® | 1.10 | 1.10 | 1.10 | 1.10 | 1.10 |
| Linoleic Acid | 0.10 | 0.10 | 0.10 | 0.10 | 0.10 |
| Stearic Acid | 0.10 | 0.10 | 0.10 | 0.10 | 0.10 |
| Vitamin E Acetate | 0.10 | 0.10 | 0.10 | 0.10 | 0.10 |
| Cholesterol | 0.25 | 0.25 | 0.25 | 0.25 | 0.25 |
| Aristoflex HMB ® | 0.25 | 0.25 | 0.25 | 0.25 | 0.25 |
| Dimethicone | 0.25 | 0.25 | 0.25 | 0.25 | 0.25 |
| Sodium Lactate | 0.10 | 0.10 | 0.10 | 0.10 | 0.10 |
| Glydant Plus ® | 0.20 | 0.20 | 0.20 | 0.20 | 0.20 |
| Fragrance | 0.15 | 0.15 | 0.15 | 0.15 | 0.15 |
| Sodium Citrate | 0.10 | 0.10 | 0.10 | 0.10 | 0.10 |
| DL Panthenol | 0.10 | 0.10 | 0.10 | 0.10 | 0.10 |
| Disodium EDTA | 0.10 | 0.10 | 0.10 | 0.10 | 0.10 |
| Triethanolamine | 0.05 | 0.05 | 0.05 | 0.05 | 0.05 |
| Water | Bal. | Bal. | Bal. | Bal. | Bal. |

### EXAMPLE 3

This Example illustrates a five minute moisture mask. Table III illustrates various related formulas.

TABLE III

| | Sample (Weight %) | | | | |
|---|---|---|---|---|---|
| Ingredient | 3A | 3B | 3C | 3D | 3E |
| Cyclopentasiloxane | 12.00 | 9.50 | 6.75 | 5.10 | 10.89 |
| SD Alcohol 38B (Menthol | 6.00 | 3.00 | 3.00 | 8.00 | 4.00 |
| Denatured) | | | | | |
| Glycerin | 2.00 | 3.00 | 1.00 | 2.00 | 0.50 |
| Butylene Glycol | 2.00 | 2.00 | 2.00 | 2.00 | 2.00 |
| Apricot Kernel Oil | 1.00 | 3.00 | 1.00 | 3.00 | 2.00 |
| Shea Butter | 0.75 | 1.00 | 0.50 | 0.50 | 1.00 |
| Cholesterol | 0.25 | 0.25 | 0.25 | 0.25 | 0.25 |
| Aristoflex HMB ® | 0.25 | 0.25 | 0.25 | 0.25 | 0.25 |
| Dimethicone | 0.25 | 0.25 | 0.25 | 0.25 | 0.25 |
| Aristoflex AVC ® | 1.25 | 1.25 | 1.25 | 1.25 | 1.25 |
| Glydant Plus ® | 0.20 | 0.20 | 0.20 | 0.20 | 0.20 |
| DL Panthenol | 0.10 | 0.10 | 0.10 | 0.10 | 0.10 |
| Disodium EDTA | 0.10 | 0.10 | 0.10 | 0.10 | 0.10 |
| Citric Acid | 0.10 | 0.10 | 0.10 | 0.10 | 0.10 |
| Titanium Dioxide | 0.10 | 0.10 | 0.10 | 0.10 | 0.10 |
| Aloe Vera | 0.10 | 0.10 | 0.10 | 0.10 | 0.10 |
| Pentylene Glycol | 0.10 | 0.10 | 0.10 | 0.10 | 0.10 |
| Phytantriol | 0.10 | 0.10 | 0.10 | 0.10 | 0.10 |
| Safflower Oil Phospholipid | 0.50 | 0.50 | 0.50 | 0.50 | 0.50 |
| Sunflower Seed Oil | 0.10 | 0.10 | 0.10 | 0.10 | 0.10 |
| Linoleic Acid | 0.10 | 0.10 | 0.10 | 0.10 | 0.10 |
| Stearic Acid | 0.10 | 0.10 | 0.10 | 0.10 | 0.10 |
| Vitamin E Acetate | 0.10 | 0.10 | 0.10 | 0.10 | 0.10 |
| Sodium Lactate | 0.10 | 0.10 | 0.10 | 0.10 | 0.10 |
| Beta Glucan | 0.10 | 0.10 | 0.10 | 0.10 | 0.10 |
| Oat Extract | 0.10 | 0.10 | 0.10 | 0.10 | 0.10 |
| Fragrance | 0.15 | 0.15 | 0.15 | 0.15 | 0.15 |
| Vitamin A Palmitate | 0.01 | 0.01 | 0.01 | 0.01 | 0.01 |
| Hyaluronic Acid | 0.01 | 0.01 | 0.01 | 0.01 | 0.01 |
| Water | Bal. | Bal. | Bal. | Bal. | Bal. |

### EXAMPLE4

A clinical study is herein reported which evaluated the effect of formulas of the present invention on reducing the size of facial pores. The study utilized a half face study design in which subjects had two different products applied to each side of their face by a clinician. A trained grader evaluated 22 female panelists. Visual evaluation for visible pores on the cheeks was conducted by using a grading scale based on a pore ruler. The pore ruler is a set of 10 images starting at 1 (very few visibly large pores) and increasing by whole points to 10 (many visibly large pores). The clinical grading scale is identified in the Table IV below. In addition to cheek pore grading, visual evaluation for visible pores on the nose was also performed using the following grading scale detailed in the Table below.

TABLE IV

| Grade | Description |
|---|---|
| 1 | None to very visible pore present |
| 2 | Slight amount of visible pores present of small size |
| 3 | Slight to moderate amount of visible pores present and/or small |
| | to moderate size |
| 4 | Moderate amount of visible pores present and/or moderate to |
| | large size |
| 5 | Extreme amount of visible pores present and/or large size |

Panelist portrait close-ups were captured using a Nikon D1 camera and 105 mm-macro lens. Panelist positioning and repositioning was managed with the Canfield Positioning System.

Each panelist had product applied to both sides of their face by a clinician. Three cotton rounds were loaded with 1.0 cc of test product. The cotton rounds were used to apply product to three sections of the face. One cotton round was used on half the forehead for 5 seconds, a second cotton round was used on half of the nose and upper cheek for 5 seconds and a third cotton round was used on half of the chin and lower cheek for 5 seconds. This study involved a single application of the test products.

Data was entered on an Excel® spreadsheet. Thereupon the mean, standard deviation and Student's t-Test were calculated. The level of significance was set prior to the test at 0.05. Digital images of the panelists' “T Zone” were captured with the camera in a horizontal/landscape orientation. Image size and quality was set to camera maximum of 2000 pixels wide by 1312 pixels high. The images were analyzed for pore size, visibility and contrast using a IDL 6.0 macro.

The test products were the formulas described in Example 1, sample 1B with 15% menthol denatured Alcohol and Sample 1E with 30% menthol denatured alcohol. Results are recorded in the Table below.

### Visual Grading of Cheek Pore Size (Mean Data)

| | | 30 Min. Post | Change from | |
|---|---|---|---|---|
| Sample | Baseline | Treatment | Baseline | p-Value |
| 1E | 5.82 | 5.32 | −0.50 | 0.00* |
| 1B | 5.82 | 5.27 | −0.55 | 0.00* |

*Indicates statistically significant difference from baseline.

Both Sample 1E and 1B revealed a significant decrease in cheek pore visibility 30 minutes post treatment. There was no significant differences noted between the two samples. There were no changes in the nose pore size 30 minutes post treatment for either of the samples tested.

Table V below reports on Sebumeter data on changes in sebum levels.

TABLE V
Sebumeter Data

| | | 30 Min. Post | Change from | |
|---|---|---|---|---|
| Sample | Baseline | Treatment | Baseline | p-Value |
| 1E | 135.50 | 105.95 | −29.55 | 0.03* |
| 1B | 127.27 | 94.05 | −33.23 | 0.01* |

Sample 1E exhibited a significant reduction in sebum levels 30 minutes post treatment. This reflected a 21.80% reduction from baseline. Sample 1B also significantly reduced skin surface oil 30 minutes post treatment. There was a 26.11% reduction from baseline for this formula.

What is claimed is:

1. A hydroalcoholic cosmetic composition comprising:
(i) from about 1 to about 60% denatured ethyl alcohol by weight of the composition and containing from about 0.3 to about 3% menthol by weight of the alcohol;
(ii) from about 0.5 to about 80% humectant by weight of the composition;
(iii) from about 0.00001 to about 3% preservative by weight of the composition; and
(iv) from about 10% to about 85% water by weight of the composition.

2. The composition according to claim 1 wherein the denatured ethyl alcohol is present in an amount from about 5% to about 25% by weight of the composition.

3. The composition according to claim 1 wherein the denatured ethyl alcohol is present in an amount from about 10% to about 40% by weight.

4. The composition according to claim 1 wherein menthol is present from about 1.3 to about 2% by weight of the denatured ethyl alcohol.

5. The composition according to claim 1 further comprising a solubilizer which is a polypropylene glycol copolymer of a C₈-C₂₄ fatty alcohol or acid.

6. The composition according to claim 5 wherein the polypropylene glycol copolymer is PPG-2-lsoceteth-20 Acetate.

7. The composition according to claim 1 which is transparent or translucent.

8. The composition according to claim 7 which is a gel having suspended therein from 1 to 1,000 gas bubbles per cc³, the bubbles having an average diameter ranging from about 0.001 to about 2 mm.

9. The composition according to claim 1 wherein the humectant is selected from the group consisting of glycerol, propylene glycol, dipropylene glycol, polypropylene glycol, polyethylene glycol, sorbitol, hydroxypropyl sorbitol, hexylene glycol, 1,3-butylene glycol, 1,2,6-hexane triol, ethoxylated glycerol, propoxylated glycerol, maltitol, isoprene glycol and mixtures thereof.

10. A method for reducing size of skin pores comprising topically applying to an external surface of a face composition comprising:
(i) from about 1 to about 60% denatured ethyl alcohol by weight of the composition and containing from about 0.3 to about 3% menthol by weight of the alcohol;
(ii) from about 0.5 to about 80% humectant by weight of the composition;
(iii) from about 0.00001 to about 3% preservative by weight of the composition; and
(iv) from about 10% to about 85% water by weight of the composition.
